(19) United States
(12) Patent Application Publication
(10) Pub. No.: US 2018/0369134 A1
(43) Pub. Date: Dec. 27, 2018

(54) Compositions and Methods of Transdermal Delivery for Therapeutic Agents

(71) Applicant: Charles L. CAVALLINO, San Diego, CA (US)

(72) Inventors: Charles L. Cavallino, San Diego, CA (US); Boris Gites, San Diego, CA (US); Jim Polansky, San Diego, CA (US)

(21) Appl. No.: 15/781,731

(22) PCT Filed: Dec. 3, 2016
(86) PCT No.: PCT/US2016/064866

§ 371 Date: Jun. 14, 2018

Related U.S. Application Data
(60) Provisional application No. 62/264,093, filed on Dec. 7, 2015.

(51) Int. Cl. A61K 9/00; A61K 47/44; A61K 47/12; A61K 9/06; A61K 9/107; A61K 31/166

(52) U.S. Cl. CPC A61K 9/0014; A61K 47/44; A61K 31/166; A61K 9/06; A61K 9/107; A61K 47/12

(57) ABSTRACT

The present invention is a composition for transdermal delivery of a therapeutic agent and methods of treating a medical condition utilizing the composition. The composition comprises a natural oil, or first oil, of about 40% to about 75% w/w of the composition and contains about 3% to 11% behenic acid, a second oil of about 2% to about 15% w/w of the composition and contains oleic and/or palmitic acids selected from the group consisting of Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, marula oil, neem oil, baobab oil, rosehip oil, and cocoa butter and an emulsifying wax of about 9% to about 32% w/w of the composition.

### CROSS-REFERENCE TO RELATED APPLICATIONS

This patent application claims priority to PCT patent application serial no. PCT/US2016/64866 filed 3 Dec. 2016 which claims priority to U.S. provisional patent application Ser. No. 62/264,093 filed 7 Dec. 2015.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates to the field of transdermal delivery of therapeutic agent for the treatment of medical conditions.

### BACKGROUND OF THE INVENTION

Transdermal administration with natural oils offers an alternative pathway for systemic drug delivery with advantages over conventional chemical routes. The stratum corneum forms a formidable barrier to dangerous exogenous molecules and it is this important protection mechanism that hinders the perecutaneous penetration of most drugs. Some chemical skin penetration enhancers are currently employed by the pharmaceutical industry and utilized in a variety of transdermal products. However, many of these are associated with irritation and toxic effects. Consequently, there has been a need for the discovery of new, safe and effective skin penetration enhancers. Because of this, penetration enhancers from natural origins have become popular. The hope has been that certain natural oils from renewable resources will provide bens over their synthetic counterparts. L. T. Fox et al. (Molecules 16:10507-10540, 2011) discusses some of the transdermal drug delivery enhancement by compounds of natural origin such as Niaouli oil, Eucalyptus oil, Alpinia oxyphylla oil, Terpentine oil, Sweet basil oil, Tulsi oil, Cardamon oil, Peppermint oil, Fennel oil and Black cumin oil. While each has provided some enhancement of drug delivery, they are not optimal transdermal delivery vehicles by themselves. In view of this, a number of formulations have been prepared containing a variety of natural oils in specific compositions as delivery vehicles. U.S. Pat. No. 6,383,495 discloses a composition for transdermal delivery containing two or more plant extracts from Tridax procumbens, Tagetes erecta, moringa oleifera, ocimum snatum, aloe vera and gum olibanum. U.S. Pat. No. 6,528,075 discloses a composition containing a long chain oil of moringa and a tocopherol for use in pharmaceutical products such as anti-acne agents. However, while these compositions have shown some effectiveness in the proscribed applications there is still a need for a composition with an ability to enhance the percutaneous penetration of a variety of therapeutic drugs.

### SUMMARY OF THE INVENTION

The present invention is a composition for transdermal delivery of a therapeutic agent. The composition comprises a natural oil or first oil of about 40% to about 75% w/w of the composition containing about 3% to 11% behenic acid, a second oil of about 2% to about 15% w/w of the composition containing oleic and/or palmitic acids, and selected from the group consisting of: Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil, cocoa butter and an emulsifying wax having a concentration of about 9% to about 32% w/w of the composition. The first oil may also be from about 45% to about 70% w/w of the composition or about 55% w/w of the composition. The second oil may also be from about 5% to about 10% w/w of said composition or about 10% w/w of said composition. Further the stearic acid NF of the second oil may range from about 3% to about 7% w/w of said composition. The emulsifying wax may be bees wax and may range from about 2% to about 10% w/w of the composition or about 3% to about 7% w/w of the composition.

In another embodiment of this aspect of the invention the composition may further comprise a fragrance.

In another aspect of the present invention the composition further comprises a therapeutic agent of about 1% to about 20% w/w of the composition, about 5% to about 15% w/w of the composition or about 5% to about 10% w/w of the composition. The therapeutic agents may be utilized for the treatment of a condition selected from the group consisting of chronic pain, a musculoskeletal disorder, a blood disorder, a central nervous system disorder and a cardiovascular disease. In one embodiment the therapeutic agent is a PDE5 inhibitor or an agent for the treatment insect bites or sun burn. The therapeutic agents that may be utilized with the present invention may have at least one benzene ring moiety and are about 50% to about 99% non-polar.

Another aspect of the present invention is a pharmaceutical composition comprising a natural oil or first oil of about 45% to about 75% w/w of the composition containing about 3% to 11% behenic acid, a second oil of about 2% to about 15% w/w of the composition containing oleic and/or palmitic acids, and selected from the group consisting of: Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, cocoa butter and an emulsifying wax having a concentration of about 9% to about 32% w/w of the composition and a therapeutic agent having a concentration of about 1% to about 20% w/w of the composition.

Yet another aspect of the invention is a method of treating a medical condition comprising the step of administering to a subject an amount of the composition above topically at a location close to or in the proximity of the medical condition.

### BRIEF DESCRIPTION OF THE DRAWINGS

None

### DETAILED DESCRIPTION OF THE INVENTION

Transdermal drug delivery offers advantages over oral administration. These advantages include: variable concentration levels in the serum are avoided; first-pass metabolism is substantially reduced; and less frequent dosing is required. Other advantages include: accessibility of the skin; large surface area for absorption and the non-invasive aspect of dermal application may increase patient compliance.

The skin is the largest organ of the body and serves as a protection layer of the underling tissues from exogenous molecules as well as from mechanical and radiation-induced injuries. The external surface of the skin, the epidermis, consists of keratinized squamous epithelium. Just below this layer, is a thick layer of dense fibroelastic connective tissue covering a subcutaneous layer (the hypodermis) comprising variable amounts of adipose tissue. Consequently, the dermis poses a unique barrier to drug delivery. The observed low permeability has been mainly attributed to the stratum corneum; the outermost layer of the skin which serves as the rate-limiting lipophilic barrier against the uptake of drugs.

The stratum corneum is several micrometers thick and is composed of flattened densely packed and tightly joined cells having severely limited intercellular space through which molecules can diffuse. The major lipids found in this layer include cholesterol and fatty acids. Because of this the stratus corneum is very selective with respect to the type of molecules it permits to be transported through the skin. Consequently, drugs must often be of extremely low molecular weight for effective delivery. Some skin penetration enhancers that have been used in the past include dimethylsulfoxide (DMSO), ethanol, propylene glycol, oleic acid, urea, dimethylacetamide and dimethylformamide (DMF). However, many of these are associated with irritation and toxic effects. Because of this, penetration enhancers from natural origins have been investigated and while each has provided some enhancement of drug delivery, they are not optimal transdermal delivery vehicles by themselves. Consequently, the present invention provides compositions for transdermal delivery of a therapeutic agent comprising a natural oil or first oil of about 45% to about 75% w/w of the composition containing about 3% to 11% behenic acid, a second oil of about 2% to about 15% w/w of the composition containing oleic and/or palmitic acids, and selected from the group consisting of: Tamanu oil, Argan oil, shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil, cocoa butter and an emulsifying wax having a concentration of about 9% to about 32% w/w of the composition.

1. Preparing Oil

Oils may be prepared in a variety of ways including cold pressing ground seeds and pressing of roasted seeds ground before pressing. Cold pressing to extract oil from seed involves the grinding of fresh fruit or seeds of the oil producing plant followed by compressing the ground seed in a mechanical press. Because these process of grinding can produce heat through friction the temperature of grinding is controlled so that it does not exceed 120° F. (49° C.). Other types of oil such as for example, peanut oil are best prepared by being roasted before being ground and pressed to eliminate specific allergens and molds that can produce toxins. This process may be performed by a screw press or spiral oil press machine. A number of screw press machines are available for example the Guangxin XZYX70 1.3 ton/day capacity (Mianyang Guangxin machinery of Grain and Oil Processing Co. Ltd. Of Shanghai, China), the HDC 15 800 Kg/hour (Wuhan HDC Technology Co. Ltd., of Shanghai, China) and the LY-109 3-5 Kg/hour (Zhengzhou Leyisi Trade Co. Ltd. Shanghai, China).

### A. First Oil

The present invention is compositions and methods of using those compositions for transdermal delivery wherein the composition comprises a first oil containing behenic acid. Oils that contain this acid include moringa oil (Ben oil), rapeseed (canola) oil and peanut oil. Oils such as these are commonly present in a daily diet. However, behenic acid is poorly absorbed and is a cholesterol-raining saturated fatty acid in humans. Consequently, oils that contain this acid are not recommended for dietary intake by individuals with high cholesterol.

Moringa oil is obtained from the fruit and leaves of Moringa oleifera, a widely cultivated species of the genus Moringa, which is the only genus in the family Moringaceae. This plant is native to the southern foothills of the Himalayas in northwestern India, where its young seed pods and leaves are used as vegetables. M oleifera is a fast-growing, deciduous tree with hanging fruit of 20-45 cm size which hold dark brown, globular seeds of about 1 cm in diameter. When cold pressing the seed of M. oleifera, a shelling machine is often used to remove the seed coats and the fresh seeds are poured into a mechanical cold press machine that crushes and pressed the seed producing oil from one outlet and a press cake from another. Approximately, 1 Kg of seed produces about 120 mL of cold pressed oil. Once extracted the oil is allowed to sit for sedimentation. The clear oil is then decanted off of the sediment.

Moringa oil may also be obtained from roasted seed. In this process the Moringa seeds are placed in a skillet and stirred until they turn a dark brown. After cooling the seeds are crushed thoroughly and placed in a pot of water that has been brought to a boil. Seeds should be boiled for about 10-15 minutes to release their oil. After straining and sitting overnight, the moringa oil may be skimmed from the surface of the water. Extraction efficiency should be about 65%.

The concentration of the behenic acid containing oil ranges from about 40% to about 75%, about 45% to about 70%, or about 55% to about 65% w/w of the composition. This oil may naturally contain about 3% to about 11%, about 5% to about 10% or about 7% to about 9% behenic acid. Alternatively, if the behenic acid is below the desired amount, oils of varying concentrations of behenic acid may be mixed to achieve the desired concentration, or the oil may be supplemented with behenic acid to bring the total concentration to the desired level. Behenic acid may be purchased from commercial suppliers such as Shanghai MOLBAS Technology, Co. (Shanghai, China), Parchem, Inc. (New Rochelle, N.Y.) or Sigma-Aldrich (St. Louis, Mo.).

Moringa oil can also be purchased commercially from Jedwards International, Inc. (Braintree, Mass.), Healing Moringa Tree, (San Bernardino Calif.) or Moringa Farms, Inc., (Sherman Oaks, Calif.).

### B. Second Oil

The second oil is provided in the composition to assist the penetration efficiency of the first oil. These oils contain oleic and/or palmitic acids at concentrations of about 2% to about 15%, about 5% to about 12% or about 7% to about 10% w/w of the composition. A variety of oils that have this concentration of oleic and/or palmitic acids may be used and include, for example, Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil and cocoa butter. To obtain the desired increase in penetration efficiency or to optimize transdermal delivery of a particular therapeutic agent one skilled in the art may combine the behenic acid containing oil with a second oil and measure penetration through the skin using a variety of methods known in the art (see Pharm Res. 30(4):1099-1109, 2013).

Tamanu oil is produced from the nut kernels of the Tamanu Tree, Calophyllum inophyllum, an evergreen tree and a member of the Mangosteen Family. This tree is actually indigenous to the tropical areas of South East Asia but it especially flourishes in tropical countries such as the exotic Melanesian islands of Vanuatu and in Polynesian islands such as Tahiti. The pale-colored nut kernels are laid out on racks to dry for 2 months. During this process, these kernels turn a brownish-red color. The oil is often extracted by cold-pressing and filtration. Tamanu oil can be purchased commercially from Organic Infusions, Inc. (Camarillo, Calif.), Vitacost (Boca Raton, Fla.) and The Vitamin Shoppe (North Bergen, N.J.).

Argan oil is obtained from the kernels of the argan tree, Argania spinosa, which is endemic to Morocco. The fruit is small and round, oval, or conical and encased in a thick peel with fleshy pulp. The hard-shelled nut represents about 25% of the weight of the fresh fruit. Each nut contains one to three argan oil-rich kernels and depending on the extraction method can yield from 30 to 50% of the oil in the kernels. To extract the kernels, the fleshy pulp is removed mechanically if the fruit is fresh or can be removed by hand if the fruit is open air dried. The nuts are then cracked by hand and the kernels extracted. This can be an arduous time-consuming and labor intensive process. The kernels are gently roast kernels. The roasted kernels are allowed to cooled and ground. The brown-colored mash when pressed expels pure, unfiltered argan oil. The argan oil is left to rest about two weeks so that suspended solids can settle to the bottom of the container. The clearer argan oil may be further filtered, depending on the required clarity and purity. Argan Oil can be purchased commercially through Bulk Apothecary (Aurora, Ohio), Jedwards International, Inc. (Braintree, Mass.) and Shea Terra Organics (Sterling, Va.).

Pecan oil is an edible pressed oil extracted from the pecan nut rich in monounsaturated fats, specifically oleic acid and contains linoleic acid (36.6%), palmitic acid (7.1%), stearic acid (2.2%) and linolenic acid (1.5%). Prior to extraction, the nuts are lightly roasted and ground. Mechanical extraction methods are then used to remove the oil. Pecan oil can be purchased commercially at Jedwards International, Inc. (Braintree, Mass.), Kinloch Plantation Products (Winnsboro, La.) and Delta Pecan Orchard (Indianola Miss.).

Canola refers to both an edible oil (also known as canola oil) produced from the seed of any of several varieties of the rape plant, and to those plants, namely a cultivar of either rapeseed (Brassica napus) or field mustard/turnip rape (Brassica rapa subsp. oleifera, syn. B. campestris). Canola oil is made by slightly heating and then crushing the seed. Almost all commercial canola oil is then refined using hexane. Finally, the Canola oil is refined using water precipitation and acid, “bleaching” with clay, and deodorizing using steam distillation. About 43% of a seed is oil. About 23 Kg of rapeseed makes 10 L of canola oil. Canola oil can be purchased commercially through local super markets or at suppliers such as Jedwards International, Inc. (Braintree, Mass.),

Olive oil is a fat obtained from the fruit of Olea europaea; family Oleaceae, a traditional tree crop of the Mediterranean Basin. The oil is produced by grinding olives and extracting the oil by mechanical or chemical means. Green olives usually produce more bitter oil, and overripe olives can produce oil that is rancid, so for good extra virgin olive oil care is taken to make sure the olives are perfectly ripened. One method includes grinding the olives into a paste. After grinding, the olive paste is spread on fiber disks, which are stacked on top of each other in a column, then placed into the press. Pressure is then applied onto the column to separate the vegetal liquid from the paste. This liquid still contains a significant amount of water. Traditionally the oil was shed from the water by gravity (oil is less dense than water). This very slow separation process has been replaced by centrifugation, which is much faster and more thorough. The centrifuges have one exit for the (heavier) watery part and one for the oil.

Neem oil is a vegetable oil pressed from the fruits and seeds of Azadirachta indica, an evergreen tree which is endemic to the Indian subcontinent and has been introduced to many other areas in the tropics. Neem oil may be produced by grinding the seed kernels in a blender or small food processor into a mash. The mash is placed in cheese cloth and hung above a large mouthed container. Water is poured through the cheese cloth and collected in the container. This is repeated and the neem oil is skimmed off the top of the water solution.

Baobab is the common name of a genus of trees Adansonia. There are eight species. Six species live in the drier parts of Madagascar, two in mainland Africa, and one in Australia. Boabab oil is made from seeds extracted from the baobab fruit. The hard outer coating of the seed capsule is cracked and the kernels removed. The kernels are then washed to remove their powder coating and air dried in the sun. The air dried kernels are then cold pressed to extract the Baobab oil.

In another more modern method, steel drum mills grind the olives to a paste which is then stirred slowly for 20 to 30 minutes in a container (malaxation), where the microscopic oil drops unite into bigger drops, which facilitates the mechanical extraction. The paste is then pressed by centrifugation; the water is thereafter separated from the oil in a second centrifugation step. In some cases, the produced oil is also filtered to eliminate remaining solid particles that may reduce the shelf life of the product. Canola oil can be purchased commercially through local super markets or at suppliers such as Baker & Olive. (Encinitas, Calif.).

Small seed oils produced from edible seeds such as poppy seeds obtained from the seeds of Papaver somniferum, sesame seeds obtained from Sesamum indicum (syn. Sesamum orientate), grape seeds from grapes, sun flower seeds from Helianthus annuus, pumpkin seed usually from the Cucurbita pepo subsp. pepo var. ‘styriaca’, also known as var. oleifera, sea buckhorn seeds derived from species of the genus Hippophae, most commonly from Hippophae rhamnoides, borneo tallow nut from the fruit of genus Shorea, native to Sarawak, Borneo, Java Malaya and the Philippines, soybean from Glycine max, macadamia nuts obtained from the fruit of Macadamia integrifolia, hemp seed obtained from Cannabis sativa, Cannabis indica or Cannabis ruderalis, maracuja seed from passion fruit, manila seed from the Sclerocarya birrea, from the Anacardiaceae family, and rosehip seeds from Rosa moschata or Rosa rubiginosa in the southern Andes or from Rosa canina, which grows in many regions of the world including South Africa and Europe may be extracted from fresh or roasted seeds using a number of screw press machines available commercially. The majority of these oils can be purchased from Parchem, Inc. (New Rochelle, N.Y.).

Shea butter is an off-white or ivory-colored fat extracted from the nut of the African shea tree, Vitellaria paradoxa. Shea butter is a triglyceride derived mainly from stearic acid and oleic acid. The traditional method of preparing unrefined shea butter consists of removing the outer pulp, drying the seed and then removing the hard outer shell. The nut is then crushed and roasted. The roasted shea nuts are ground into a smoother paste, water is gradually added and the paste is mixed. The paste is kneaded by hand in large basins and water is gradually added to help separate out the butter oils. As they float to the top, the butter oils, which are in a curd state, are removed and excess water squeezed out. The butter oil curds are then melted and boiled to remove any remaining water. The shea butter is ladled from the top of the water and put in cool places to harden. Shea butter can be purchased commercially through, Cleopatra's Choice (Plainfield, Ind.), Bulk Apothecary (Aurora, Ohio) and BGLH (Chicago, Ill.).

Cocoa butter is prepared by first lightly roasting the whole bean with shell at a temperature that does not exceed 135° F. The beans are then de-shelled and fed into a grinder. The nibs are ground into a fine powder. This powder is heated until the fat in the nibs is melted into chocolate liquor made of roughly equal parts cocoa solids and cocoa butter. The liquor is filtered to remove cocoa solids. The oil eventually cools into a solid yellow block of cocoa butter.

C. Oleic acid and Palmitic Acid

The concentration of the triglycerides of oleic acid and palmitic acid may be determined by a variety of methods known in the art. For example, the boronnitrifluoride method of Hisil (Instrumental Analysis Techniques Ege Univ. Engineer Fac, Publ. Nu 55 Bornova-Izmir utilizing gas chromatography such as an Hewlett-Packard 6890 Series II gas chromatograph may be used to identify these triglycerides. Conditions utilized for analysis vary depending on the oil being analyzed. A standard fatty acid methyl ester mixture (Sigma-Aldrich Co., St. Louis Mo.) may be used to confirm peaks. In addition, commercial mixtures of fatty acid methyl esters may be used as reference data for the relative retention times. Quantitative analysis of the fatty acids may be performed using the heptadecanoic acid methyl ester as an internal standard.

The approximate compositions of these fatty acids in the second oils are shown in the Table 1.

TABLE 1
Fatty acid compositions by percentage w/w of a variety of natural oils.

| | | | | | | | Poly | |
|---|---|---|---|---|---|---|---|---|
| | | | | | | | unsaturated | |
| | | | | | | Mono | | Alpha |
| | Saturated | | | | | unsaturated | Linoleic | Linolenic |
| | Capric | Lauric | Myristic | Palmitic | Stearic | Oleic | Acid | Acid |
| | Acid | Acid | Acid | Acid | Acid | Acid | (ω6) | (ω3) |
| Oil or Fat | C10:0 | C12:0 | C14:0 | C16:0 | C18:0 | C18:1 | C18:2 | C18:3 |
| Baobab Oil | — | — | — | 18-30 | 2-9 | 30-42 | 20-35 | 1-3 |
| Hemp seed | — | — | — | 6-7 | 2-3 | 10-13 | 54-57 | 16-20 |
| Oil | | | | | | | | |
| Neem Oil | — | — | — | 16-33 | 9-24 | 25-54 | 6-16 | — |
| Macadamia | — | — | — | 19 | — | 60 | 1-3 | 1-2 |
| nut | | | | | | | | |
| Oil | | | | | | | | |
| Maracuja Oil | — | — | — | 5-18 | 1-3 | 10-22 | 77 | — |
| Marula Oil | — | — | — | 9-12 | 5-8 | 70-78 | 4-7 | 0.1-0.7 |
| Borneo | — | — | 3 | 26 | 14 | 47 | 3 | 1 |
| Tallow | | | | | | | | |
| nut Oil | | | | | | | | |
| Rosehip Oil | — | — | — | 3.4-4.4 | 1-3 | 14-16 | 43-46 | 31-34 |
| Sea Buckhorn | — | — | — | 7-31 | — | 17-20 | 40 | 30-36 |
| seed Oil | | | | | | | | |
| Pumpkin seed | — | — | 0.1-0.3 | 13-18 | 5-9 | 17-40 | 18-63 | 0.3-0.8 |
| Oil | | | | | | | | |
| Poppy seed | — | — | — | 10 | 2 | 11 | 5 | — |
| Oil | | | | | | | | |
| Canola Oil | — | — | — | 4 | 2 | 62 | 22 | 10 |
| Cocoa Butter | — | — | — | 25 | 38 | 32 | 3 | — |
| Grape seed | — | — | — | 8 | 4 | 15 | 73 | — |
| Oil | | | | | | | | |
| Olive Oil | — | — | — | 13 | 3 | 71 | 10 | 1 |
| Peanut Oil | — | — | — | 11 | 2 | 48 | 32 | — |
| Sesame Oil | — | — | — | 9 | 4 | 41 | 45 | — |
| Shea nut | — | 1 | — | 4 | 39 | 44 | 5 | |
| Soybean Oil | — | — | — | 11 | 4 | 24 | 54 | 7 |
| Sunflower | — | — | — | 7 | 5 | 19 | 68 | 1 |
| Oil* | | | | | | | | |
| Tamanu Oil | — | — | — | 12 | 13 | 34 | 38 | — |

Certain fatty acid compositions may be desired for a particular formulation. Consequently the composition of the formulation may be adjusted by incorporating one or more of the second oils to achieve the desired composition. Alternatively, if a single second oil is desired over a combination of second oils the fatty acid of interest may be purchased in pure form commercially (Sigma-Aldrich Co., St. Louis, Mo. or Acme Hardesty Co., Blue Bell, Pa.) and added to the desired oil to adjust the concentration of that fatty acid to the desired amount.

2. Emulsifying Wax

Emulsifying wax is utilized to provide a desired viscosity, maintain a desired homogeneity and desired consistency for dermal application. The concentration of the emulsifying wax may be from about 9% to about 32% w/w, about 13% to about 25% w/w or about 20% w/w. It may be prepared by combining a wax material (e.g., bees wax or vegetable wax) with a detergent such as sodium dodecyl sulfate or polysorbates causing the oil and water to bind together into a smooth emulsion. The emulsifying wax may contain a variety of compounds depending on the intended use. For cosmetic formulations, specifically National Formulary “NF”, the emulsifying was may generally contain cetearyl alcohol, Polysorbate 60, PEG-150 stearate, and Steareth-20 and is an effective thickener that assists in stabilizing emulsions and formulations. The amount of bees wax in the emulsifying wax may range from about 2% to about 10% w/w, 3% to about 7% w/w or about 5% w/w. The amount of stearate and/or stearic acid NF in the emulsifying wax may range from about 1% to about 10% w/w, about 3% to about 7% w/w or about 5% w/w.

3. Therapeutic Agents

A variety of therapeutic agents for treating a variety of conditions may be incorporated into the transdermal delivery composition of the present invention. Particularly preferred therapeutic agents are those that are about 50% to about 99% non-polar and have at least one benzene ring moiety. Conditions that may be treated by these therapeutic agents include for example, chronic pain, musculoskeletal disorders, blood disorders, central nervous system disorders or cardiovascular disease. One particularly preferred group of therapeutic agents that may be incorporated into the transdermal delivery compositions of the present invention are phosphodiesterase type 5 “PDE5” inhibitors. These drugs block the degradative action of cGMP-specific PDE5 on cyclic GMP in the smooth muscle cells lining the blood vessels supplying the corpus cavernosum of the penis and are used in the treatment of erectile dysfunction. Because PDE5 is also present in the arterial wall smooth muscle within the lungs, PDE5 inhibitors have also been explored for the treatment of pulmonary hypertension, a disease in which blood vessels in the lungs become overloaded with fluid, usually as a result of failure of the left ventricle of the heart. A pharmaceutical composition comprising a therapeutic drug and the transdermal delivery composition of the present invention may contain from about 1% to about 20% w/w of a therapeutic agent.

Another group of therapeutic agents that may be incorporated into the transdermal delivery compositions of the present invention are salicylates. The use of salicylates, specifically acetylsalicylic acid (aspirin) and salicylic acid has been known since antiquity; remedies have been discovered as far back as ancient Egypt, Sumer and Assyria. There are multiple mechanisms of action for salicylates; for example aspirin non-selectively and irreversibly inhibits different cyclooxygenase isozymes: COX-1 and COX-2. Normally COX produces prostaglandins, most of which are pro-inflammatory, and thromboxanes, which promote clotting. Similarly, salicylic acid has also been shown to activate adenosine monophosphate-activated protein kinase (AMPK), which may play a role in the anticancer effects of the compound. There are also additional effects that AMPK activation helps create anti-diabetic effects. Aspirin has been shown effective in treatment of pain, headache, fever, swelling and inflammation, heart attack/stroke, cancer prevention and other vital uses. Salicylic acid has been shown to be effective for the treatment of seborrhoeic dermatitis, acne, psoriasis, calluses, corns, keratosis pilaris, acanthosis nigricans, ichthyosis, and warts. A pharmaceutical composition comprising a therapeutic drug and the transdermal delivery composition of the present invention may contain from about 1% to about 20% w/w of a therapeutic agent.

Pain management is a broad field in which there are many topical applications. Diclofenac has shown to treat pain, dysmenorrhea and inflammatory disorders such as arthritis, rheumatoid arthritis, polymyositis, dermatomyositis, osteoarthritis, dental pain, TMJ pain, spondylarthritis, ankylosing spondylitis, gout attacks, and pain management in cases of kidney stones and gallstones. Research indicates that diclofenac acts by inhibiting prostaglandin synthesis by inhibiting cyclooxygenase (COX), which results in the reduced formation of prostaglandins, thromboxanes and prostacyclin. A pharmaceutical composition comprising a therapeutic drug and the transdermal delivery composition of the present invention may contain from about 1% to about 20% w/w of a therapeutic agent.

Transdermal products can also be used for cosmetic indications such as cellulite removal or masking. Classic cellulite remedies include using caffeine, a psychoactive drug and a central nervous system stimulant that is found in many popular beverages such as coffee and cola and using retinol, a type of vitamin A which is convertible to other types of vitamin A and retinyl ester for the storage of vitamin A in animals. Together, when combined and applied topically, caffeine and retinol immediately improve the smooth appearance of skin and repair cell structure over time to ensure cellulite does not return. Medical evidence has shown that caffeine is vasoconstrictor, or narrowing of the blood vessels, but increases basal metabolic rate. It is believed that caffeine, or siloxanetriol alginate caffeine, spikes circulation and shrinks fat cells short-term when applied topically to areas afflicted with cellulite. Additionally, retinol works by stimulating collagen growth and normalizing the skin re-production process over time. A composition comprising of these agents and the transdermal delivery composition of the present invention may contain from about 1% to about 20% w/w of a therapeutic agent.

Further topical application includes vasodilator medication which slows or stops hair loss such as Rogaine®. The mechanism of action is not clearly understood; a theory of why hair regrowth happens is that by widening blood vessels and opening potassium channels, more oxygen, blood, and nutrients flow to the follicle. A pharmaceutical composition comprising a therapeutic drug and the transdermal delivery composition of the present invention may contain from about 1% to about 20% w/w of a therapeutic agent.

Other skin conditions such as poison oak/ivy, eczema, dermatitis, allergies, rash, itching, and redness can be treated with topical corticosteroids. Cortisol, being one of those steroids, counteracts insulin to stimulate gluconeogenesis, or formation of glucose, to aid in metabolism of fat, protein and carbohydrates in people who normally do not produce enough cortisol. This process also activates anti-stress and anti-flammatory functions. In order to treat skin allergies, some of which are caused by stress, cortisol reduces overall B-cell-mediated antibody activity to relieve skin irritation. A composition comprising of these agents and the transdermal delivery composition of the present invention may contain from about 1% to about 20% w/w of a therapeutic agent.

One of the most common active ingredients for insect repellant is DEET, or diethyltoluamide. Applied topically or to clothing, DEET repels mosquitos, ticks, fleas and other biting insects. Recent evidence has shown that mosquitoes and other insects dislike the scent of DEET, where special antennal sensilla are activated by the drug repelling the insect from approaching the body. Concentrations of DEET products can vary all the way up to 100%, however, varying concentrations are directly proportional to hours of protection; for example, 20-34% can offer about 3-6 hours of protection. A pharmaceutical composition comprising a therapeutic drug and the transdermal delivery composition of the present invention may contain from about 1% to about 20% w/w of a therapeutic agent.

4. Formulation

A variety of methods known in the art may be used to prepare an emulsion with the first and second oils. For example, a basic emulsion may be prepared using the formula of 1 part emulsifier, 3 parts oil or butter and 6 parts water. The emulsifier may be a combination of beeswax 80%, borax 10%, and lecithin 10%. In one example, the emulsion may be prepared by heating the waxes, oils/butters and lecithin together. In a separate container, combine the water and borax and heat. When the waxes have fully melted and the borax/water solution is heated begin whipping the water phase while slowly adding thin stream of the oil/was mixture. After the ingredients are fully combined, continue to whip the mixture approximately fill five minutes until the mixture becomes thick and opaque.

A variety of methods know to those in the art may be utilized to combine the therapeutic agent or agents of choice into the transdermal delivery composition of the present invention (Applied Pharmaceutics in Contemporary Compounding R. Shrewsburg Morton Publishing Englewood, Colo.). In this particular case, two solutions are prepared while mixing under heated conditions. The first, waxes are melted into a liquid state; while in the second solution the active ingredient is added to the mixture of the two oils. Once the active is dissolve the wax solution is added to the active mixture and mixed until homogenous. Stearic acid and fragrance are then added, mixed until homogenous and then left to cool to room temperature.

5. Use

A medical condition may be treated with a formulation of a desired drug and the transdermal delivery composition of the present invention by administering to a subject an amount of a composition comprising a first oil, a second oil, an emulsifying wax and a therapeutic agent of an effective concentration for single or multiple applications topically at a location close to or in the proximity of the medical condition. Two examples of the types of applications that may be utilized with the present invention include a single and multiple dose regimens. In the single dose regimen, an amount of the formulation is measured with the help of a measuring device; recommendations given by the doctor and/or pharmacist may supersede written instructions, and rubbed into the affected area at proscribed intervals. In the multiple dose regimen, a first application is applied to the affected area using a minimal amount of cream. The cream is rubbed on and around the affected area to ensure the cream penetrates the skin. This is repeated within 10-15 minutes of the last application until the desired relief is obtained.

We claim:

1. A composition for transdermal delivery of a therapeutic agent comprising:
a first oil, wherein said first oil is a natural oil of about 40% to about 75% w/w of said composition and containing about 3% to 11% behenic acid;
a second oil, wherein said second oil is about 2% to about 15% w/w of said composition and containing oleic and/or palmitic acids, further wherein said second oil is selected from the group consisting of:
Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil, cocoa butter; and
an emulsifying wax having a concentration of about 9% to about 32% w/w of said composition.

2. The composition according to claim 1, wherein said first oil is about 45% to about 70% w/w of said composition.

3. The composition according to claim 1, wherein said first oil is about 55% w/w of said composition.

4. The composition according to claim 1, wherein said second oil is about 5% to about 10% w/w of said composition.

5. The composition according to claim 1, further comprises stearic acid NF of about 1% to about 10% w/w of said composition.

6. The composition according to claim 7, wherein said stearic acid NF is about 3% to about 7% w/w of said composition.

7. The composition according to claim 1, wherein said emulsifying wax is bee's wax of about 2% to about 10% w/w of said composition.

8. The composition according to claim 10, wherein said bees wax is about 3% to about 7% w/w of said composition.

9. The composition according to claim 1, further comprising a fragrance.

10. The composition according to claim 1, further comprising therapeutic agent of about 1% to about 20% w/w of said composition.

11. The composition according to claim 14, wherein said therapeutic agent is about 5% to about 15% w/w of said composition.

12. The composition according to claim 14, wherein said therapeutic agent is about 5% to about 10% w/w of said composition.

13. The composition according to claim 14, wherein said therapeutic agent is an agent for the treatment of a condition selected from the group consisting of chronic pain, a musculoskeletal disorder, a blood disorder, a central nervous system disorder and a cardiovascular disease.

14. The composition according to claim 14, wherein said therapeutic agent is a PDE5 inhibitor.

15. The composition according to claim 14, wherein said therapeutic agent is an agent for the treatment insect bites or sun burn.

16. The composition according to claim 14, wherein said therapeutic agent is an agent having at least one benzene ring moiety and is about 50% to about 99% non-polar.

17. A pharmaceutical composition comprising: a first oil, wherein said first oil is a natural oil of about 40% to about 75% w/w of said composition and containing about 3% to 11% behenic acid; a second oil, wherein said second oil is about 2% to about 15% w/w of said composition and containing oleic and/or palmitic acids, further wherein said second oil is selected from the group consisting of: Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil, cocoa butter; an emulsifying wax having a concentration of about 9% to about 32% w/w of said composition; and a therapeutic agent having a concentration of about 1% to about 20% w/w of said composition.

18. A method of treating a medical condition comprising the step of administering to a subject an amount of a composition comprising a first oil, wherein said first oil is a natural oil of about 45% to about 75% w/w of said composition and containing about 3% to 11% behenic acid; a second oil, wherein said second oil is about 2% to about 15% w/w of said composition and containing oleic and/or palmitic acids, further wherein said second oil is selected from the group consisting of: Tamanu oil, Argan oil, Shea butter, Pecan oil, canola oil, olive oil, poppy seed oil, sesame oil, sea buckthorn oil, grape seed oil, sunflower oil, macadamia oil, hemp oil, soybean oil, pumpkin seed oil, Borneo tallow nut oil, maracuja oil, manila oil, neem oil, baobab oil, rosehip oil, cocoa butter; an emulsifying wax having a concentration of about 9% to about 32% w/w of said composition; and a therapeutic agent having a concentration of about 1% to about 20% w/w of said composition topically at a location close to or in the proximity of said medical condition.
